(19) United States
(12) Patent Application Publication
(10) Pub. No.: US 2017/0367660 A1
(43) Pub. Date: Dec. 28, 2017

(54) OUTPUT METHOD, OUTPUT DEVICE, AND COMPUTER-READABLE RECORDING MEDIUM

(71) Applicant: FUJITSU LIMITED, Kawasaki-shi (JP)

(72) Inventors: Takashi SHIMADA, Shinagawa (JP); Kosei Takano, Fujimino (JP); Masayoshi Hoshiya, Yokohama (JP); Masatsugu Isogai, Yokohama (JP)

(73) Assignee: FUJITSU LIMITED, Kawasaki-shi (JP)

(21) Appl. No.: 15/698,698

(22) Filed: Sep. 8, 2017

Related U.S. Application Data
(63) Continuation of application No. PCT/JP2016/050887, filed on Jan. 13, 2016.

(30) Foreign Application Priority Data
Mar. 12, 2015 (JP) 2015-049835

(51) Int. Cl. A61B 5/00; A61B 5/024; A61B 5/022; A61B 5/01; G06F 19/00; A61B 5/08

(52) U.S. Cl. CPC A61B 5/7246; G06F 19/322; A61B 5/4815; A61B 5/082; A61B 5/0022; A61B 5/022; A61B 5/01; A61B 5/024

(57) ABSTRACT

A server device acquires a monitoring result of a specific type of vital sign of a specific user and identification information of a first monitoring device from the first monitoring device that monitors the specific type of vital sign. The server device acquires a monitoring result of a specific type of vital sign of the specific user and identification information of a second monitoring device from the second monitoring device that monitors the specific type of vital sign. For a time series fluctuation of the specific type of vital sign of the specific user, when a combination of the monitoring result of the first monitoring device and the monitoring result of the second monitoring device is to be output, the server device outputs the monitoring result of the first monitoring device and the monitoring result of the second monitoring device in a distinguishable manner.

### CROSS-REFERENCE TO RELATED APPLICATION

This application is a continuation of International Application No. PCT/JP2016/050887, filed on Jan. 13, 2016 which claims the benefit of priority of the prior Japanese Patent Application No. 2015-049835 filed on Mar. 12, 2015, the entire contents of which are incorporated herein by reference.

### FIELD

The embodiment discussed herein is related to an output method, an output device, and an computer-readable recording medium.

### BACKGROUND

In recent years, various types of health monitoring devices are used under various environments to monitor biological condition of users and manage the health of the users based on monitoring results thereof. Examples of the health monitoring devices include, but not limited to, devices such as a pulse rate meter that monitors a pulse and a sphygmomanometer that monitors blood pressure. These devices are made by different manufacturers, and various models are produced by the same manufacturer. For example, in case of a pulse rate meter, there are a contact-type pulse rate meter for monitoring a pulse in a contact-state i.e. in contact with a user's body and a non-contact type pulse rate meter for monitoring a pulse in a non-contact state i.e. without contacting a user's body.

There is also a case where the pulse of a specific user is monitored by using both the contact type pulse rate meter and the .non-contact type pulse rate meter simultaneously.

### Patent Literature 1: Japanese Laid-open Patent Publication No. 2009-136377

However, it is demanded to monitor a specific type of vital sign of a specific user, for example, a pulse, using the health monitoring devices and to compare time series fluctuations of monitoring results; of the health monitoring devices related to the pulse of the specific user.

### SUMMARY

According to an aspect of an embodiment, an output method for executing a process includes acquiring a monitoring result of a specific type of vital sign of a specific user and identification information of a first monitoring device from the first monitoring device that monitors the specific type of vital sign. The method includes acquiring a monitoring result of a specific type of vital sign of the specific user and identification information of a second mentoring device from the second monitoring device that monitors the specific type of vital sign. The method includes transmitting the monitoring result of the first monitoring device and the monitoring result of the second monitoring device in a distinguishable manner, for a time series fluctuation of the specific type of vital sign of the specific user, when a combination of the monitoring result of the first monitoring device and the monitoring result of the second monitoring device is to be transmitted.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is an explanatory diagram illustrating an example of a monitoring result output system according to a present embodiment;

FIG. 2 is an explanatory diagram illustrating an example of a health monitoring device;

FIG. 3 is an explanatory diagram illustrating an example of a server device;

FIG. 4 is an explanatory diagram illustrating an example of a terminal device;

FIG. 5 is an explanatory diagram illustrating an example of a record structure of a user DB;

FIG. 6 is an explanatory diagram illustrating an example of a record structure of an information DB;

FIG. 7 is an explanatory diagram illustrating an example of a display screen of time series fluctuation graphs on a display unit of the terminal device;

FIG. 8 is a flowchart illustrating an example of a processing operation of the server device related to time series fluctuation output processing;

FIG. 9 is an explanatory diagram illustrating an example of the display screen of time series fluctuation graphs on the display unit of the terminal device;

FIG. 10 is an explanatory diagram illustrating art example of the display screen of time series fluctuation graphs on the display unit of the terminal device;

FIG. 11 is an explanatory diagram illustrating an example of the display screen of the time series fluctuation graph on the display unit of the terminal device; and

FIG. 12 is an explanatory diagram illustrating an example of a computer that executes an output program.

### DESCRIPTION OF EMBODIMENTS

Preferred embodiments of the present invention will be explained with reference to accompanying drawings. In addition, the embodiments are not intended to limit the disclosed technology. The embodiments explained below may be combined appropriately without containing inconsistencies.

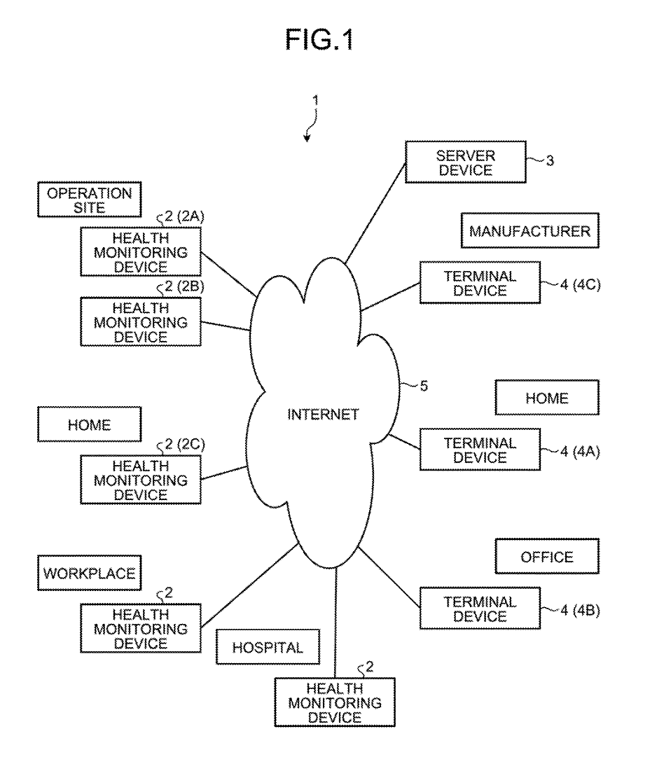

FIG. 1 is an explanatory diagram illustrating an example of a monitoring result output system 1 according to the present embodiment. The monitoring result output system 1 illustrated in FIG. 1 includes a plurality of health monitoring devices 2, a server device 3, and a plurality of terminal devices 4. The health monitoring device 2 is a device that monitors biological condition of a user placed, for example, at home, an operation site, a workplace, and a hospital. The health monitoring device 2 is a monitoring device such as a sphygmomanometer, a weight scale, a thermometer, an alcohol sensor, and a sleep monitoring device. The server device 3 makes a communication with each health monitoring device 2 via, for example, Internet 5. Besides, the server device 3 collects biological data of each user acquired by the health monitoring device 2 via the Internet 5.

The terminal device 4 is a terminal device such as a computer disposed at an individual or a company licensed to use the monitoring result output system 1, for example, at a user's home, a user's office, and a manufacturer that manufactures the health monitoring device 2. As the terminal device 4, for example, the terminal device disposed at the user's home is referred to as 4A, the terminal device disposed at the user's office is referred to as 4B, and the terminal, device disposed at the manufacturer of the health monitoring device 2 is referred to as 4C. The terminal device 4 makes a communication with the server device 3 via, for example, the Internet 5.

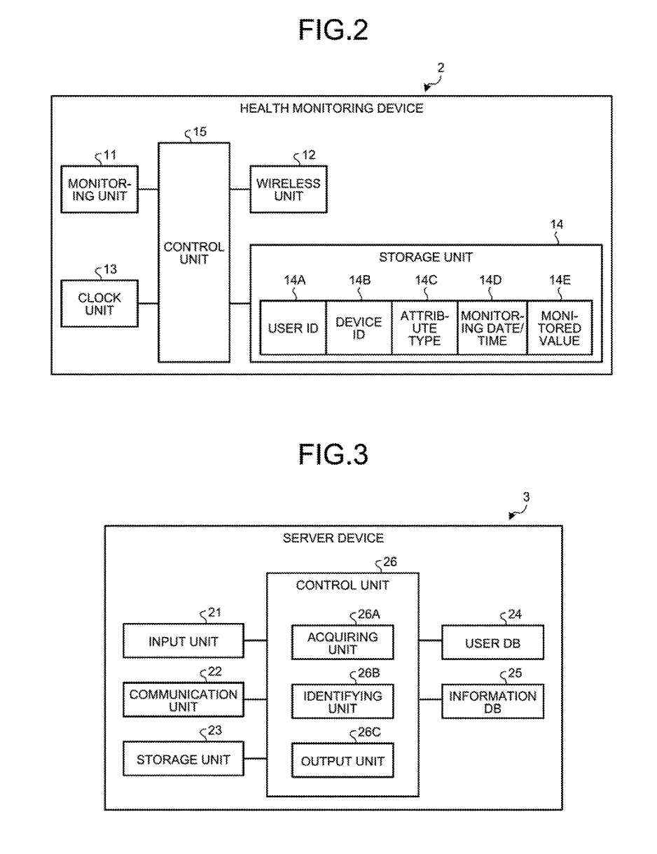

FIG. 2 is an explanatory diagram illustrating an example of the health monitoring device 2. The health monitoring device 2 illustrated in FIG. 2 includes a monitoring unit 11, a wireless unit 12, a clock unit 13, a storage unit 14, and a control unit 15. The monitoring unit 11 monitors biological condition of the user. When the health monitoring device 2 is a pulse rate meter, the monitoring unit 11 is a contact type pulse rate meter, such as an ear clip type, for monitoring a user's pulse in contact with a user's body or a non-contact type pulse rate meter for monitoring a user's pulse with millimeter waves, microwaves, or the like without contacting the user's body. When the health monitoring device 2 is a sphygmomanometer, the monitoring unit 11 is, for example, a contact type or a non-contact type blood pressure monitoring unit that monitors the blood pressure of the user. For example, when the health monitoring device 2 is a weight, scale, the monitoring unit 11 is, for example, a contact type or a non-contact type weight scaling unit that scales the weight of the user. For example, when the health monitoring device 2 is a thermometer, the monitoring unit 11 is, for example, a contact type or a non-contact type body-temperature monitoring unit that monitors the temperature of the user. For example, when the health monitoring device 2 is a monitoring device that detects blood alcohol concentration in breath, the monitoring unit 11 is a monitoring unit that monitors the amount of alcohol in the breath to estimate the blood alcohol concentration of the user. For example, when the health monitoring device 2 is a sleep quality monitoring device, the monitoring unit 11 is a monitoring unit that monitors the quality of sleep of the user.

The wireless unit 12 is, for example, a communication interface that makes a communication with the Internet 5 in a wireless manner. When the wireless unit 12 is not built in, the health monitoring device 2 may include a communication function with the Internet 5 by using a terminal device such as a smartphone. The clock unit 13 clocks, for example, a date and time of monitoring by the monitoring unit 11. The storage unit 14 is an area that stores therein monitoring results of monitored values or the like for each monitoring date and time for each user ID for identifying the user of the health monitoring device 2.

The storage unit 14 stores a monitoring result including a device ID 14B, an attribute type 14C, a monitoring date and time 14D, and a monitored value 14E for each user ID 14A. The user ID 14A is identification information for identifying a user of the health monitoring device 2. The device ID 14B is identification information for identifying the health monitoring device 2 for, for example, each manufacturer. The device ID 14B is assumed to be stored in the storage unit 14. The attribute type 14C is monitoring type of the health monitoring device 2, for example, a data type such as pulse, blood pressure, and blood alcohol concentration. The monitoring date and time 14D is a date and time of monitoring by the monitoring unit 11 monitored by the clock unit 13. The monitored value 14E is a monitored value monitored by the monitoring unit 11.

When monitoring the biological condition of a user using the monitoring unit 11, the control unit 15 stores, in the storage unit 14, the monitoring results associated with the device ID 14B, the attribute type 14C, the monitoring date and time 14D, and the monitored value 14E of the health monitoring device 2 for each user ID 14A for identifying the user.

FIG. 3 is an explanatory diagram illustrating an example of the server device 3. The server device 3 illustrated in FIG. 3 includes an input unit 21, a communication unit 22, a storage unit 23, a user database (DB) 24, an information DB 25, and a control unit 26. The server device 3 collects biological data of users from the health monitoring devices 2 via the Internet 5.

The input unit 21 is an input interface that receives various commands. The communication unit 22 is a communication interface that communicates with, for example, the Internet The storage unit 23 is an area that stores therein various types of information such as various programs.

The user DB 24 is an area that stores therein personal information of a user by each user ID to identify the user. FIG. 5 is an explanatory diagram illustrating an example of a record structure of the user DB 24. The user DB 24 illustrated in FIG. 5 is an area that stores therein personal information associated with a user name 24B, a gender 24C, and an age 24D for each user ID 24A. The user ID 24A is identification information for identifying, for example, the user. The user name 24B is, for example, the user's first and last name. The gender 24C is, for example, the sex of the user. The age 24D is, for example, the age and date of birth of the user.

The control unit 26 updates and registers the user ID 24A, the user name 24B, the gender and the age 24D in the user DB 24 with an input operation from, for example, the licensed terminal devices 4.

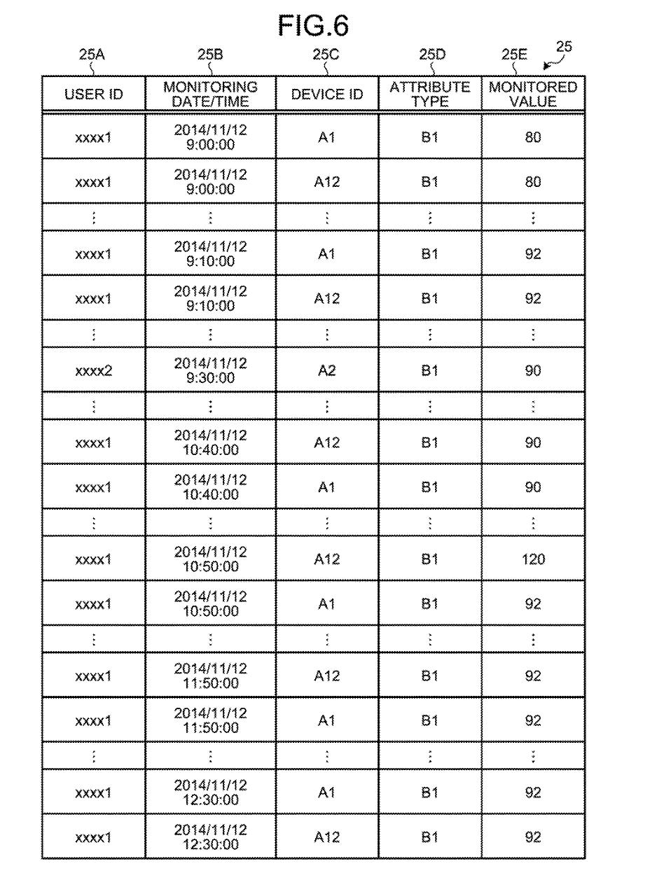

The information DB 25 is a storage area that stores therein monitoring results of biological condition of the user monitored by each health monitoring device 2 by each user ID 25A to identify the user. FIG. 6 is an explanatory diagram illustrating an example of a record structure of the information DB 25. The information DS 25 stores monitoring results associated with a device ID 25C, an attribute type 25D, and a monitored value 25E for each user ID 25A and monitoring date and time 25B. The user ID 25A is, for example, an ID for identifying the user. The monitoring date and time 25B is the monitoring date and time monitored by the health monitoring device 2. The device ID 25C is identification information for identifying the health monitoring device 2 being a monitoring source. The attribute type 25D is a type of data monitored by the health monitoring device 2 being the monitoring source. The monitored value 25E is the monitoring result monitored by the health monitoring device 2 being the monitoring source.

The control unit 26 collects monitoring results for each user from the health monitoring devices 2 via the communication unit 22. Then the control unit 26 stores, in the information DB 25, the user ID, the monitoring date and time, the device ID, the attribute type, and the monitored value within the collected monitoring results as the user ID 25A, the monitoring date and time 25B, the device ID 250, the attribute type 25D, and the monitored value 25E respectively.

For example, it is assumed, that a health monitoring device 2A is a contact type pulse rate meter disposed at an operation site, a health monitoring device 2B is a non-contact type pulse rate meter disposed at the same operation site, and a health monitoring device 2C is a contact type pulse rate meter disposed at home. Besides, the device ID 25C of the health monitoring device 2A is referred to as “A1”, the device ID 25C of the health monitoring device 2B is referred to as “A12”, and the device ID 25C of the health monitoring device 20 is referred to as “A2”. Furthermore, for example, the attribute type 25D being pulse is referred to as “B1”.

The health monitoring device 2A disposed at the operation site monitors the pulse of a user through the monitoring unit 11, and stores the monitoring results including the user ID “xxxx 1”, the device ID “A1”, the attribute type “B1”, the monitored value, and the monitoring date and time in the storage unit 14. The health monitoring device 2A then transmits the monitoring results stored in the storage unit 14 to the server device 3 at a predetermined frequency via the Internet 5 through the wireless unit 12.

The health monitoring device 23 disposed at the same operation site also monitors the pulse of the user through the monitoring unit 11, and stores the monitoring results including the user ID “xxxx 1”, the device ID “A12”, the attribute type “B1”, the monitored value, and the monitoring date and time in the storage unit 14. The health monitoring device 2B then transmits the monitoring results stored in the storage unit 14 to the server device 3 at the predetermined frequency via the Internet 5 through the wireless unit 12.

The health monitoring device 2C disposed at home monitors the pulse of a user through the monitoring unit 11, and stores the monitoring results including the user ID “xxxx 2”, the device ID “A2”, the attribute type “B1”, the monitored value, and the monitoring date and time in the storage unit 14. The health monitoring device 2C then transmits the monitoring results stored in the storage unit 14 to the server device 3 at the predetermined frequency vis the Internet 5.

To collect monitoring results from the health monitoring devices 2, the control unit 26 in the server device 3 stores the monitoring results in the information DB 25. The control unit 26 manages the respective data in the user DB 24 and in the information DB 25 linked to the user ID 24A (25A).

The control unit 26 retrieves the output program stored in the storage unit 23 and executes the retrieved output program, and thereby executes the output program as a function. The control unit 26 includes, as a functional configuration, an acquiring unit 26A, an identifying unit 26B, and an output unit 26C. The acquiring unit 26A collects monitoring results from the health monitoring devices 2, and stores the collected monitoring results in the information DB 25. The identifying unit 26B determines whether monitoring results of the specific user ID 25A and the attribute type 25B exist in the information DB 25. When monitoring results of the specific user ID 25A and the attribute type 25D exist, the identifying unit 26B identifies the monitoring results of the specific user ID 25A and the attribute type 25D. Besides, the identifying unit 26B determines whether monitoring results with different device IDs 25C exist among the identified monitoring results. The identifying unit 26B identifies the monitoring results with different device IDs 25C among the identified monitoring results.

The output unit 26C generates time series fluctuation data indicating time series fluctuation of a monitored value for each device ID 25C based on the monitored value 25E and the monitoring date and time 25B among the identified monitoring results. The time series fluctuation data is the data indicating the time series fluctuation of the monitoring values based on the monitored value and the monitoring date and time among the monitoring results. Furthermore, the output unit 26C transmits the generated time series fluctuation data for each device ID 25C to a predetermined destination. The predetermined destination is the destination information on the terminal device 4 licensed, to connect to the monitoring result output system 1.

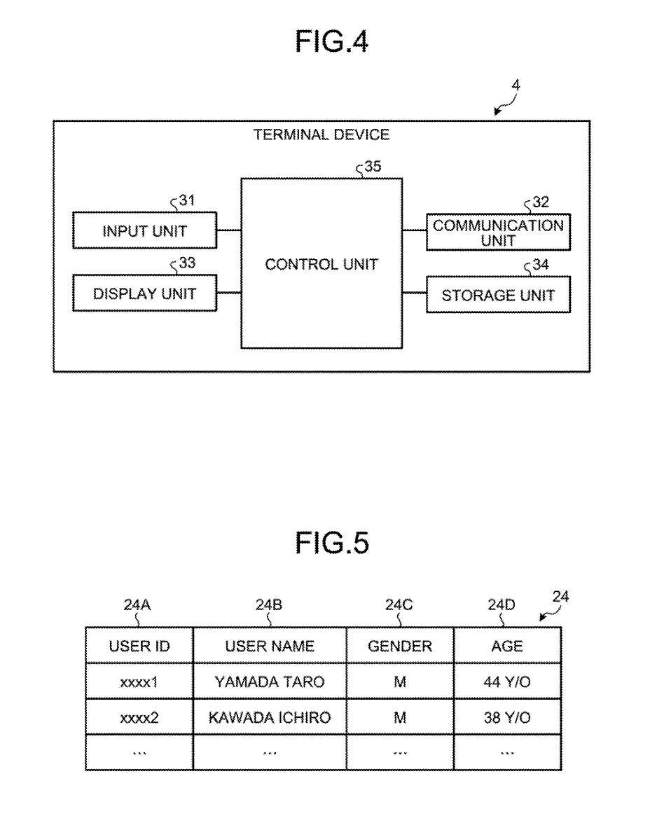

FIG. 4 is an explanatory diagram illustrating an example of the terminal device 4. The terminal device 4 is, for example, a computer or a smartphone disposed at the home of the user of the health monitoring device 2, a computer at the workplace that uses the monitoring results of the user of the health monitoring device 2, and a computer of the manufacturer of the health monitoring device 2, which are licensed to connect to the monitoring result, output system 1.

The terminal device 4 illustrated in FIG. 4 includes an input unit 31, a communication unit 32, a display unit 33, a storage unit 34, and a control unit 35. The input unit 31 is an input interface that receives various commands. The communication unit 32 is a communication interface that makes a communication, for example, via the Internet 5. The display unit 33 is an output interface that displays various types of information. The storage unit 23 is an area that stores therein various types of information. The control unit 35 controls the entire terminal device 4.

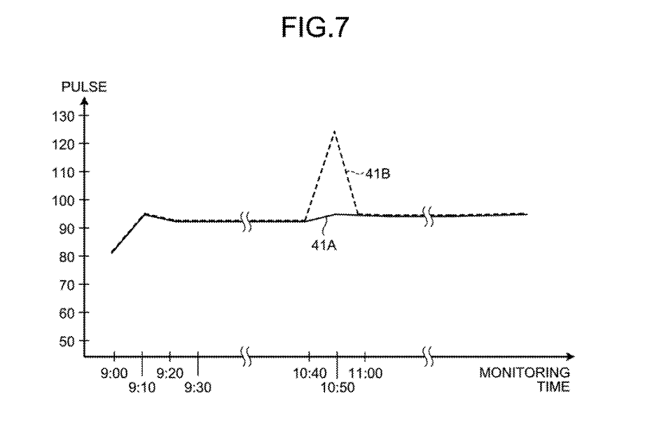

To receive user's time series fluctuation data from the server device 3, the control unit 3b of the terminal device 4 displays a time series fluctuation graph for each device ID on the screen of the display unit 33 based on the time series fluctuation data. FIG. 7 is an explanatory diagram illustrating an example of a display screen of time series fluctuation graphs on the display unit 33 of the terminal device 4.

What appears on the display screen illustrated in FIG. 7 is a time series fluctuation graph 41A, by “solid line”, indicating a time series fluctuation of the monitored value of the user ID “xxxx 1” monitored by the health monitoring device 2A of the device ID “A1”, based on Pulse plotted on the vertical axis and Monitoring Time: plotted on the horizontal axis. What, appears also on the display screen is a time series fluctuation graph 41E, by “dotted line”, indicating a time series fluctuation of the monitored value of the user ID “xxxx 1” monitored by the health monitoring device 2B of the device ID “A12”. As a result, the user of the terminal device 4 can distinguish chronologically the monitoring results monitored by the different health monitoring devices 2A and 2B among the monitoring results of the specific user and the specific type (pulse).

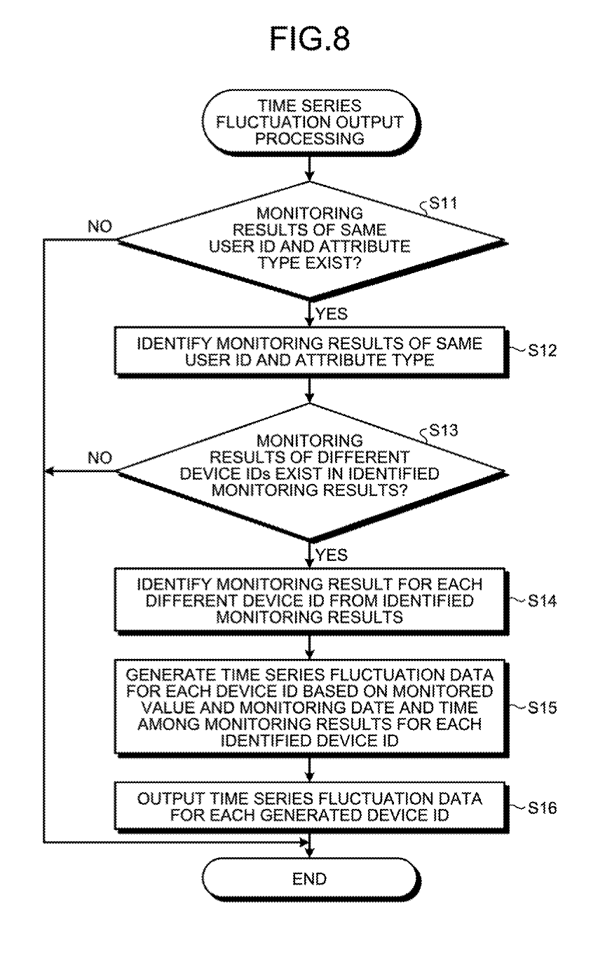

The operation of the monitoring result output system 1 according to present embodiment will be explained next. FIG. 8 is a flowchart illustrating an example of the processing operation of the server device 3 related to time series fluctuation output processing. The time series fluctuation output processing illustrated in FIG. 8 is processing of generating and outputting time series fluctuation data indicating time series fluctuation of the monitored values monitored by different health monitoring devices 2, from the information DB 25, among the monitoring results of the same user and the same attribute type.

As illustrated in FIG. 8, the identifying unit 26B of the control unit 26 in the server device 3 determines whether monitoring results of the same user ID 25A and attribute type 25D exist in the monitoring results stored in the information DB 25 (Step S11). When monitoring results of the same user ID 25A and attribute type 25D exist (Yes at Step S11) the identifying unit 25B identifies the monitoring results of the same user ID 25A and attribute type 25D from the information DB 25 (Step S12). The identifying unit 26B identifies, for example, the monitoring result of the attribute type “B1” of the user ID “xxxx 1” from the information DB 25,

The identifying unit 26B determines whether monitoring results including different device IDs 25C exist in the identified monitoring results (Step S13). When monitoring results including different device IDs 25C exist (Yes at Step S13), the identifying unit 26B identifies the monitoring results including the different device IDs 25C in the monitoring results identified at Step S12 (Step S14). The identifying unit 26B identifies, for example, the monitoring results of the device IDs “A1” and “A12” among the monitoring results of the attribute type “B1” of the user ID “xxxx 1” from the information DB 25.

The output unit 26C generates time series fluctuation data for each device ID 25C based on the monitored value 25E and the monitoring date and time 25B among the monitoring results of each device ID 25C identified at Step 214 (Step S15). In other words, the output unit 26C generates respective time series fluctuation data of the device IDs “A1” and “A12” among the monitoring results of the attribute type “B1” of the user ID “xxxx 1”. The output unit 26C outputs the time series fluctuation data for each device ID 250 (Step S16), and ends the processing operation illustrated in FIG. 8.

When no monitoring result of the same user ID and attribute type exists (No at Step S11) or when no monitoring result of different device IDs 250 exists (No at Step S13), the identifying unit 26B ends the processing operation illustrated in FIG. 8.

The control unit 26 identifies the monitoring results of the same user, the same attribute type, and different device IDs from the information DB 25, generates time series fluctuation data for each device ID based on the monitored values and the monitoring date and time of the identified monitoring results, and outputs the time series fluctuation data.

When the time series fluctuation data for each device ID is to foe transmitted, the server device 3 outputs the time series fluctuation data to the terminal device 4 as a predetermined destination via the Internet 5. The terminal device 4 displays the time series fluctuation graphs 41A and 41B for each device 10 on the display unit 33, as illustrated in FIG. 7, based on the time series fluctuation data received from the server device 3, The predetermined, destination to which the time series fluctuation data is output includes the terminal device 4 of the user licensed to the user as a target for monitoring the time series fluctuation data, the terminal device 4 at an office that requires the monitoring results of the user, and the terminal device 4 of the manufacturer of the health monitoring device 2 which is a monitoring source of the time series fluctuation data. When the time series fluctuation data is generated, the server device 3 transmits the time series fluctuation data to the terminal devices 4 as the predetermined destination of the time series fluctuation data, for example, to the terminal device 4 of the user, the terminal device 4 of the office, and the terminal device 4 of the manufacturer.

The control unit 35 of the terminal device 4 combines the time series fluctuation graphs for each device ID based on the time series fluctuation data and displays them on the display unit 33. The control unit 35 represents the time series fluctuation graph 41A of the health monitoring device 2A in the device ID “A1” by a solid line, and represents the time series fluctuation graph 41B of the health monitoring device 2B in the device ID “A12” by a dotted line. As a result, the user of the terminal device 4 visually recognizes the solid line of the time series fluctuation graph 41A on the display screen to distinguish the time series fluctuation of the monitored value of the health monitoring device 2A with the device ID “A1” from the other. Besides, the user visually recognizes the dotted line of the time series fluctuation graph 41B on the display screen to distinguish the time series fluctuation of the monitored value of the health monitoring device 2B with the device ID “A12” from the other. That is, the user can easily compare the time series fluctuation of the monitoring results related to the specific attribute type of the specific user acquired by the different health monitoring devices 2.

When identifying the monitoring results of the different device IDs among the monitoring results of the same user ID and attribute type, the server device 3 generates time series fluctuation data of which time series fluctuation is distinguishable for each device ID based on the monitored value and the monitoring date and time for the identified device ID, and transmits the time series fluctuation data to the terminal device 4. Besides, if the terminal 4 receives the time series fluctuation data for each device ID, the terminal device 4 displays the time series fluctuation graphs in a distinguishable manner for each device ID on the screen of the display unit 33 based on the time series fluctuation data. As a result, the user of the terminal device 4 visually recognizes the time series fluctuation graphs 41A and 41B for each device ID on the display screen to recognize the time series fluctuation s of the monitoring results of the different health monitoring devices 2 for the same user and attribute type.

The server device 3 according to the embodiment identifies the monitoring results of the different device IDs among the monitoring results of the same user and attribute type, generates time series fluctuation data based on the monitored value and the monitoring date and time for each identified device ID, and transmits the data to the terminal device 4 as a predetermined destination. Then the terminal device 4 displays the time series fluctuation graphs on the display unit 33 in a distinguishable manner for each device ID based on the time series fluctuation data for each device ID. On the other hand, the server device 3 may provide the monitoring result including the monitoring value and the monitoring date and time for each identified device ID to the terminal device 4 as a predetermined destination. In this case, the terminal device 4 receives the monitoring result for each identified device ID, and generates the time series fluctuation data based on the monitored value and the monitoring date and time among the monitoring results for each device ID. As a result, the processing load can be reduced when the server device 3 generates the time series fluctuation data.

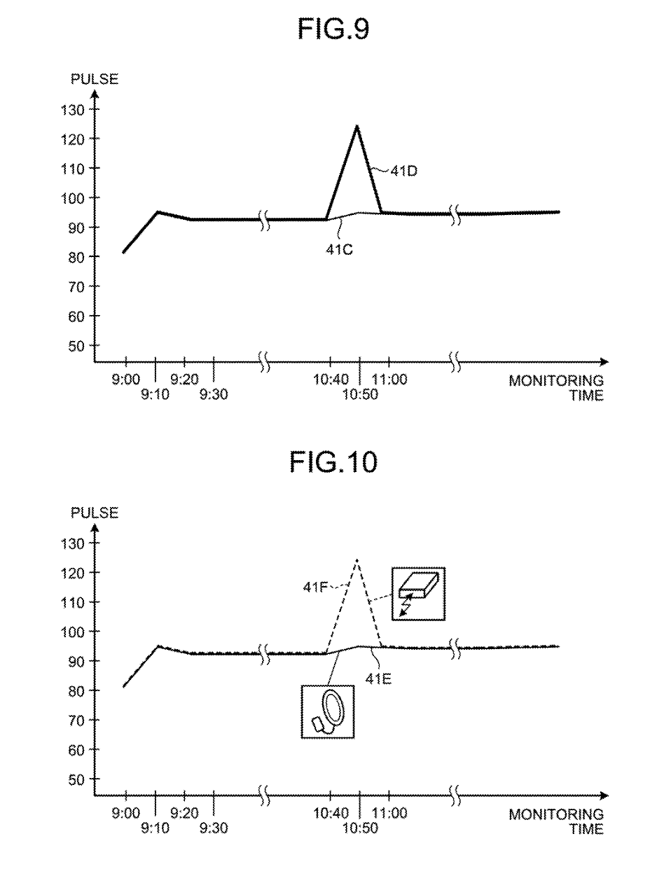

In the embodiment, as illustrated in FIG. 7, the time series fluctuation graphs 41A and 41B are displayed, in a distinguishable manner for each device ID by, for example, a line type such as a solid line and a dotted line. Also, the time series fluctuation graphs may be displayed in a distinguishable manner by changing color. FIG. 9 is an explanatory diagram illustrating an example of the display screen of time series fluctuation graphs on the display unit 33 of the terminal device 4. What is displayed in red on the display screen illustrated in FIG. 9 is a time series fluctuation graph 41C indicating a time series fluctuation of the monitoring value of the user ID “xxxx 1” monitored by the health monitoring device 2A of the device ID “A1”. What is displayed in green also on the display screen is a time series fluctuation graph 41D indicating a time series fluctuation of the monitored value of the user ID “xxxx 1” monitored by the health monitoring device 2B of the device ID “A12”. As a result, the user of the terminal device 4 can distinguish the time series fluctuation s of the monitored values, by the colors, monitored by the health monitoring devices 2.

Besides, an icon is attached to each time series fluctuation graph on the display screen so that the time series fluctuation graphs may be displayed in a distinguishable manner on the display screen. FIG. 10 is an explanatory diagram illustrating an example of the display screen of time series fluctuation graphs on the display unit 33 of the terminal device 4. What is displayed on the display screen illustrated in FIG. 10 is a time series fluctuation graph 41E indicating a time series fluctuation of the monitored value of the user ID “xxxx 1” monitored by the health monitoring device 2A of the device ID “A1” with an icon indicating “Ear clip type” as the health monitoring device 21 attached. What is also displayed on the display screen is a time series fluctuation graph 41F indicating a time series fluctuation of the monitored, value of the user ID “xxxx 1” monitored by the health monitoring device 2B of the device ID “A12” with an icon indicating a non-contact type pulse rate meter as the health monitoring device 2B attached. As a result, the user of the terminal device 4 can distinguish the time series fluctuation s of the monitored values, by the icons, monitored by the respective health monitoring devices 2.

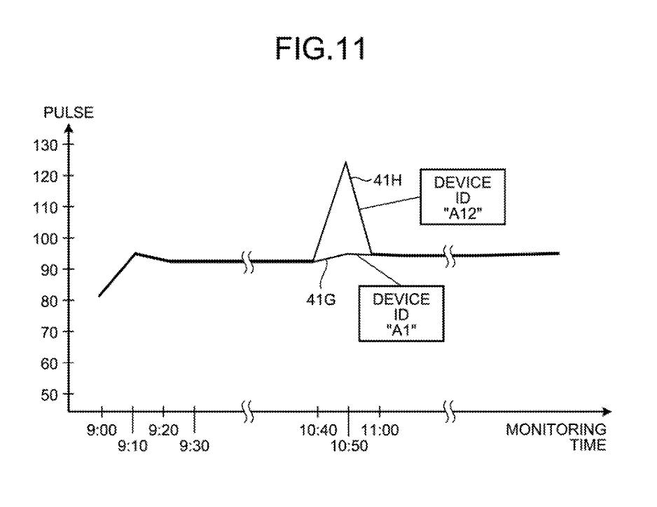

In addition, a device ID is attached to each time series fluctuation graph on the display screen, so that the time series fluctuation graphs may be displayed in a distinguishable manner on the display screen. FIG. 11 is an explanatory diagram illustrating an example of the display screen of the time series fluctuation graphs on the display unit 33 of the terminal devices 4. What is displayed on the display screen illustrated in FIG. 11 is a time series fluctuation graph 41G indicating a time series fluctuation of the monitored value of the user ID “xxxx 1” monitored by the health monitoring device 2A of the device ID “A1” with the device ID “A1” attached, What is also displayed on the display screen is a time series fluctuation graph 41R indicating a time series fluctuation of the monitored value of the user ID “xxxx 1” monitored by the health monitoring device 28 of the device ID “A12” with the device ID “A12” attached. As a result, the user of the terminal device 4 can distinguish the time series fluctuations of the monitored values, by the indication of the device IDs, monitored by the respective health monitoring devices 2.

Although the embodiment exemplifies pulse as the attribute type, the embodiment is not limited to the pulse, and various types of biological condition such as vital signs, for example, blood pressure, blood alcohol concentration, body weight, height, and body fat percentage may be used, and they can be changed if necessary.

In the embodiment, the time series fluctuation, data of the two time series fluctuation graphs are generated based on the monitored values and the monitoring date and time of the same user and attribute type obtained by, for example, two health monitoring devices 2. However, the embodiment is not limited to, for example, the two health monitoring devices 2, and it may be configured to generate three or more time series fluctuation graphs based on the monitored values and the monitoring date and time of the same user and attribute type obtained by the three or more health monitoring devices 2. As a result, the three or more time series fluctuation graphs are displayed distinguishably in the terminal devices 4, so that the monitoring results of the respective health monitoring devices 2 can be recognized in chronological order.

It may be configured that the server device 3 accepts a display request in which specific user and attribute type are specified from the terminal device 4 licensed to the user and provides the time series fluctuation data related to the monitored values of the different device IDs among the monitoring results of the specified user and attribute type to the terminal device 4 of the display request. In this case, the user of the terminal device 4 can specify an arbitrary user, an attribute type, and a device ID among the monitoring results to be displayed.

The terminal device 4 displays the time series fluctuation graphs distinguishably for each device ID on the time axis based on the monitored values and the monitoring date and time among the monitoring results. Also, time series fluctuation graphs on a dairy or monthly basis may be displayed instead of the time series fluctuation graphs on an hourly basis.

The server device 3 identifies the monitoring results of the specific user and attribute type from the information DB 25, identifies the monitoring results of the different device IDs from the identified monitoring results, and generates the time series fluctuation data based on the monitoring date and time and the monitored value among the identified monitoring results. Also, the server device 3 may identify, for example, the monitoring result of the attribute type from the information DB 25, identify the monitoring results of the different device IDs from the identified monitoring results, and generate and transmit the time series fluctuation data based on the identified monitoring results. Therefore, the method of identifying monitoring results may be changed if necessary.

In the embodiment, the health monitoring device 2 is exemplified as the device ID. Also, the embodiment is not limited to the health monitoring device 2 that monitors biological information, and it is applicable to a monitoring device that monitors, for example, atmospheric temperature. In this case, it is assumed that the user ID is set to a location ID indicating an installation location of the monitoring device, the attribute type is set to atmospheric temperature, and the device ID is set to a temperature monitoring device, and when a plurality of temperature monitoring devices are arranged in the same location, the time series fluctuation graphs of the temperature monitoring devices can be displayed in a distinguishable manner.

The server device 3 transmits the time series fluctuation data for each device ID to the terminal device 4A, as a predetermined destination, of the user's home with the user ID related to the monitoring result. Besides, when, the time series fluctuation data for each device ID is to be transmitted to the predetermined destination, the server device 3 may output the time series fluctuation data to the terminal device 4 of the manufacturer that manufactures the health monitoring device 2 of the device ID. As a result, a user of the manufacturer of the terminal device 4C can compare the time series fluctuation graph, monitored by the health monitoring device 2 of other manufacturer related to the specific attribute type of the monitored by the health monitoring device 2 of the manufacturer.

The server device 3 transmits the time series fluctuation data for each device ID to the terminal device 4, as a predetermined destination, of a licensee interested in the monitoring results of the user. As a result, the terminal device 4 of the licensee interested in the monitoring results of the user can compare the time-series fluctuation graphs of the monitoring results of different health monitoring devices 2 having the specific attribute type of the specific user.

The monitoring result output system 1 according to the embodiment allows the both devices such as the server device 3 and the terminal device 4 to communicate with each other via the Internet 5. Besides, for example, a local area network (LAN) may be used instead of the Internet 5, and the system can be changed if necessary.

In the embodiment, although the server device 3 is a computer and the terminal device 4 is a user's computer, various functions and information of the server device 3 and the terminal device 4 may be implemented by cloud computing.

The respective components of the illustrated units are not necessarily configured as physically illustrated ones. In other words, the specific mode of decentralization and integration of the units is not limited to the illustrated one. Namely, it can be configured by functionally or physically decentralizing or integrating all or part of the units in an arbitrary unit according to the various kinds of load and the use conditions.

Moreover, various processing functions performed by computers that respectively constitute the server device 3 and the terminal device 4 may execute all or arbitrary part of the functions on a central processing unit (CPU) (or microcomputer such as a micro processing unit (MPU)), a micro controller unit (MCU)), or the like. It is regardless to say that ail or arbitrary part of the various processing functions may be executed on a program analyzed and executed by the CPU (or microcomputer such as MPU and MCU) or on hardware by wired logic.

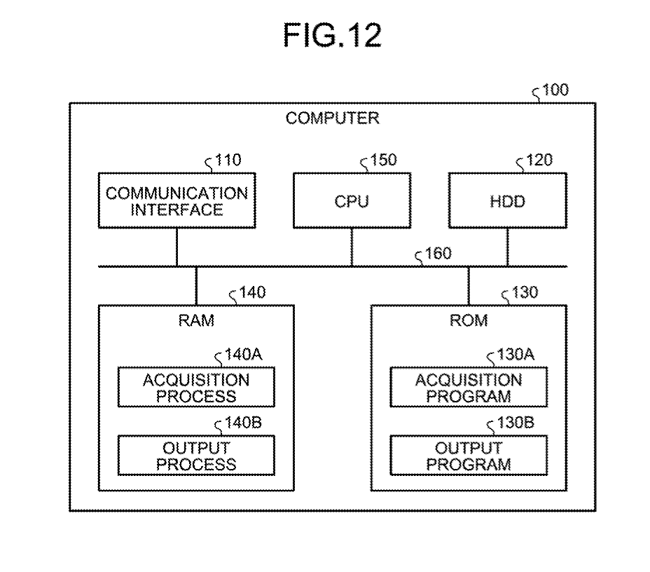

Incidentally, the various processes described in the present embodiment can be implemented by a computer executing the program prepared in advance. Therefore, an example of a computer that executes the program including the same functions as these of the embodiment will be explained below. FIG. 12 is an explanatory diagram illustrating an example of a computer 100 that executes an output program.

The computer 100 that executes the output program in FIG. 12 includes a communication interface 110, a hard disk drive (HDD) 120, a read-only memory (ROM) 130, a random access memory (RAM) 140, a CPU 150, and a bus 160.

The ROM 130 previously stores the output program performing the same function as that of the embodiment. Besides, a processing program may be recorded on a recording medium readable by a drive (not illustrated) instead of the ROM 130. The recording medium may be, for example, a portable recording medium such as a compact disk read only memory (CD-ROM), a digital versatile disk (DVD), and Universal Serial Bus (USB) memory, and a secure digital (SD) card, and a semiconductor memory such as a hard disk drive (HDD) and a flash memory. The processing program is an acquisition program 130A and an output program 130B, The acquisition program 130A and the output program 130B may be integrated or decentralized if necessary.

The CPU 150 retrieves the acquisition program 130A and the output program 130B from the ROM 130 and executes the retrieved programs. The CPU 150 orders the acquisition program 130A and the output program 130B to function as an acquisition process 140A and an output process 140B on the RAM 140.

The CPU 150 acquires a monitoring result of a specific type of vital sign of a specific user and identification information of a first monitoring device from the first monitoring device that monitors the specific type of vital sign, Besides, the CPU 150 acquires a monitoring result of a specific type of vital sign of a specific user and identification information of a second monitoring device from the second monitoring device that monitors the specific type of vital sign. For the time series fluctuation or the specific type of vital sign of the specific user, when a combination of the monitoring result of the first monitoring device and the monitoring result of the second monitoring device is to be transmitted, the CPU 150 transmits the monitoring result of the first monitoring device and the monitoring result of the second monitoring device in a distinguishable manner. As a result, the time series fluctuations s of the monitoring results of the specific type of vital sign of the specific user acquired by the different monitoring devices can easily be compared.

As one aspect of the present invention, it is possible to easily compare the time series fluctuation of the monitoring results of a specific type of vital sign of a specific user acquired by different monitoring devices.

All examples and conditional language recited herein are intended for pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the philosophy and scope of the invention.

What is claimed is:

1. An output method for executing a process, the method comprising;
acquiring a monitoring result of a specific type of vital sign of a specific user and identification information of a first monitoring device from the first monitoring device that monitors the specific type of vital sign;
acquiring a monitoring result of a specific type of vital sign of the specific user and identification information of a second monitoring device from the second monitoring device that monitors the specific type of vital sign; and
outputting the monitoring result of the first monitoring device and the monitoring result of the second monitoring device in a distinguishable manner, for a time series fluctuation of the specific type of vital sign of the specific user, when a combination of the monitoring result of the first monitoring device and the monitoring result of the second monitoring device is to be output.

2. The output method according to claim 1, further comprising:
outputting, when the time series fluctuation of the vital sign is to be output, the fluctuation result of the first monitoring device and the monitoring result of the second monitoring device in a distinguishable manner by a distinguishing mars for distinguishing between the first monitoring device and the second monitoring device,

3. The output method according to claim 1, further comprising:
outputting, when the time series fluctuation of the vital sign is to be output, the monitoring result of the first monitoring device and the monitoring result of the second monitoring device in a distinguishable manner by changing a line type or a color.

4. The output method according to claim 1, further comprising:
outputting, when the time series fluctuation of the vital sign is to be output, the monitoring result of the first monitoring device and the monitoring result of the second monitoring device in a distinguishable manner by icon for distinguishing between the first monitoring device and the second monitoring device.

5. A non-transitory computer-readable recording medium having stored therein a program that causes a computer to execute a process comprising:
acquiring a monitoring result of a specific type of vital sign of a specific user and identification information of the first monitoring device from the first monitoring device that monitors the specific type of vital sign;
acquiring a monitoring result of a specific type of vital sign of the specific user and identification information of the second monitoring device from the second monitoring device that monitors the specific type of vital sign; and
outputting the monitoring result of the first monitoring device and the monitoring result of the second monitoring device in a distinguishable manner, for a time series fluctuation or the specific type of vital sign of the specific user, when a combination of the monitoring result of the first monitoring device and the monitoring result of the second monitoring device is to be output,

6. An output device comprising:
a processor configured to:
acquire a monitoring result of a specific type of vital sign of a specific user and identification information of a first monitoring device from the first monitoring device that monitors the specific type of vital sign;
acquire a monitoring result of a specific type of vital sign of the specific user and identification information of a second monitoring device from the second monitoring device that monitors the specific type of vital sign; and
output the monitoring result of the first monitoring device and the monitoring result of the second monitoring device in a distinguishable manner, for a time series fluctuation of the specific type of vital sign of the specific user, when a combination of the monitoring result of the first monitoring device and the monitoring result of the second monitoring device is to be output.
